(19) United States
(12) Patent Application Publication
(10) Pub. No.: US 2011/0144031 A1
(43) Pub. Date: Jun. 16, 2011

(54) Pharmaceutical composition for inducing damages of endothelial cells and treating tumor and method for treating tumor by using the same

(75) Inventors: Huan-Yao Lei, Tainan City (TW); Ming-Chen Yang, Taipei City (TW)

(73) Assignee: National Cheng Kung University, Tainan City (TW)

(21) Appl. No.: 12/923,463

(22) Filed: Sep. 23, 2010

(30) Foreign Application Priority Data
Dec. 11, 2009 (TW) 098142641

(51) Int. Cl. A61K 38/16; C07K 14/42; A61P 35/00; C12N 5/00

(52) U.S. Cl. 514/19.3; 530/370; 435/375

(57) ABSTRACT

The present invention relates to a pharmaceutical composition for inducing damages of endothelial cells, a pharmaceutical composition for treating a tumor, and a method for treating a tumor by using the same. In addition, the pharmaceutical compositions for inducing damages of endothelial cells comprises: an effective amount of Concanavalin A (Con A).

### BACKGROUND OF THE INVENTION

1. Field of the Invention

The present invention relates to pharmaceutical compositions for inducing damages of endothelial cells and treating a tumor, and a method for treating a tumor by using the same and, more particularly, to a pharmaceutical composition for inducing damages of hepatic endothelial cells, a pharmaceutical composition for treating a liver tumor, and a method for treating a liver tumor by using the same.

2. Description of Related Art

Lectins are sugar-binding proteins, which are highly specific for binding to monosaccharide. Lectins also attach themselves to cells or specifically bind to sugars or sugar-containing compounds. Generally, lectins are well distributed in nature, such as plants, microorganisms, or humans, and plant seeds have been proven to contain large amounts of lectins. In addition, lectins are considered as plant defense proteins against environmental toxins. Lectins have high binding ability to monosaccharides, so they can induce cell activation, and stimulate various biological activities.

Concanavalin A (Con A) is a lectin extracted from Jack bean, Canavalia ensiformis. It is specific for binding to mannose or glucose, and induces cell aggregation. Con A is the most widely used lectin, and it has been proven that Con A can activate T cells and be used as a T-cell mitogen to induce cell proliferation. Some researches have found that Con A can induce natural killer T (NKT) cells, and then activate CD4⁺ T cells to induce hepatitis in mice (Tiegs et al., 1992, J. Clin. Invest. 90: 196-203; Kaneko et al., 2000, J. Exp. Med. 191:105-114). In addition, T-cell-dependent acute liver injury induced by Con A in mice is considered as a model of human autoimmune hepatitis, and the mechanism of T-cell-mediated acute hepatic injury is described as follow. After intravenous administration of Con A, Kupffer cells are activated and secrete TNFα, IL-12, and IL-18. T cells including NTK cells and CD4⁺ T cells are also activated, and the T cells further generate IFN-γ and activate Kupffer cells through a positive feedback system. In this mechanism, super oxide/reactive oxygen species (ROS), TNFα, and IFN-γ relate to liver apoptosis. In addition, several molecules or cells, such as IL-4, IL-6, IL-10, MIF, selectin adhesion molecule and ICAM-1, and neutrophils or T-regulatory (Treg) cells, also relate to the regulation of hepatic injury (Ksontini et al., 1998, J. Immunol. 160: 4082-4089; Massaguer et al., 2002, J. Leukoc. Biol. 72: 262-270; Bonder et al., 2004, J. Immunol. 172: 45-53; Klein et al., 2005, J. Clin. Invest. 115: 860-869; Nakajima et al., 2006, Liver Int. 26: 346-51; Zhu et al., J. Immunol. 178: 5435-5442; Erhardt et al., 2007, Hepatology 45: 475-485; Nakajima et al., 2008, Hepatology 48: 1979-1988; Jiang et al., J. Immunol. 182: 3768-3774).

In addition, Con A has been proven to induce hepatitis in SCID/NOD mice (Chang and Lei, 2008, Int. J. Immunopharmaco. Pharmacol. 21:817-26). The mechanism of the hepatitis induced by the administration of Con A into the SCID/NOD immunodeficient mice is different from that induced by the administration of Con A in to BALB/c mice. For example, the dose for the SCID/NOD mice to induce the hepatitis is larger than that for the BALB/c mice, there is no lymph node infiltration in the SCID/NOD mice, and the conditions of generating cytokine are also different. In addition, in the hepatitis induced by NKT/CD4⁺ T cells, the liver injury is induced by apoptosis. However, in the T-cell-independent acute liver injury included by Con A in SCID/NOD mice, the hepatic cell death is induced by autophagy (Chang and Lei, 2008, Int. J. Immunopharmaco. Pharmacol. 21: 817-26).

Autophagy is a catabolic process involving the degradation of a cell's own components through the lysosomal machinery. Autophagy involves the cytoplasmic balance to control the transfer of the long-lived protein. Autophagy is also related to the starvation, the tumor progression, the immune response, and the therapeutic application. In addition, autophagy may also be induced by lectins such as Con A (Kirkegaard et al., 2004, Nat. Rev. Microbiol. 2: 301-314; Kondo et al., 2005, Nat Rev Cancer 5: 726-734; Münz, 2006, Cell Microbiol. 8: 891-898; Rubinsztein et al., 2007, Nature Reviews in Drug Discovery 6: 304-312; Mizushima et al., 2008, Nature 451: 1069-75; Zhao et al., 2008, Cell Host Microbe 4: 458-469). Con A can directly induce hepatic autophagy, and also activate T cells in the immune system. In a murine in situ hepatoma model, Con A is effective in the anti-hepatoma treatment, and has dose-dependence and time-dependence (Chang et al., 2007, Hepatology 45: 286-296).

The mechanisms related to the tumors are very complex. Therefore, it is desirable to provide a pharmaceutical composition and a method using the same, which can induce damages of endothelial cells to achieve the purpose of treating a tumor.

### SUMMARY OF THE INVENTION

The inventors found that Con A can induce damages of endothelial cells to cause the destruction of blood vessels, and has effect on the treatment of tumors. Therefore, the present invention is completed based on the above findings.

The object of the present invention is to provide a pharmaceutical composition for inducing damages of endothelial cells, which can be used in the treatment of a tumor.

Another object of the present invention is to provide a pharmaceutical composition for treating a tumor, which is accomplished by the damages of endothelial cells.

A further object of the present invention is to provide a method for treating a tumor, especially for treating a liver tumor (hepatoma).

To achieve the object, the pharmaceutical composition for inducing damages of endothelial cells of the present invention comprises: an effective amount of Concanavalin A (Con A).

In addition, the pharmaceutical composition for treating a tumor of the present invention comprises: an effective amount of Con A.

Furthermore, the method of treating a tumor of the present invention comprises: administering a pharmaceutical composition to a patient, wherein the pharmaceutical composition comprises an effective amount of Con A.

According to the present invention, Con A induces damages of endothelial cells. Herein, the endothelial cells may be hepaticendothelial cells, or liver tumor endothelial cells. Preferably, the endothelial cells are hepatic vascular endothelial cells, or liver tumor vascular endothelial cells.

According to the pharmaceutical composition for treating a tumor and the method by using the same of the present invention, the tumor preferably is a liver tumor.

In addition, according to the present invention, the effective amount of Con A is 20-40 mg/kg.

According to the present invention, Con A can be extracted from a natural source, or be obtained through chemical synthesis.

Furthermore, according to the present invention, the pharmaceutical composition may further comprise IFN-γ (interferon-γ), which can enhance the effect of Con A on the autophagy of the endothelial cells. A suitable concentration of IFN-γ is 400-800 U/ml.

The hepatitis induced by Con A can be divided into T-cell-dependent hepatitis and T-cell-independent hepatitis, and the modes causing cell death of these two types of hepatitis are different. Con A induces the apoptosis process in the T-cell-dependent hepatitis, but Con A induces the autophagy process in the T-cell-independent hepatitis. However, in these two types of hepatitis, blood vessels are the first target, and the damage of endothelial cells induces cytokine-directed damages of hepatic cells. The damage of endothelial cells also induces plasma leakage and hemorrhage without cell infiltration. Hence, the damage of endothelial cells causing by Con A-induced autophagy is the prerequisite for the recruit of the inflammatory cells and the hepatic cell death.

In addition, Con A induces dose-dependent and time-dependent cell death, and LC3 (microtubule-associated protein light chain 3) and BNIP (BCL2/adenovirus E1B 19 kd-interacting protein) conversion on HMEC-1 cells. In addition, when Con A is injected to a murine model, it is found that Con A deposits on the hepatic sinusoidal endothelial cells and hepatocytes. The liver tissue is stained with anti-LC3-II (microtubule-associated protein light chain 3) antibody, and the punctate staining (an indicator of autophagy characteristics) is found to be present on the hepatocytes. In addition, according to Western blot analysis, the levels of LC3 conversion can be detected. Endothelial cells expose under many stresses, and autophagy can maintain the internal balance of endothelial cells. Under the stimulation of Con A, autophagy can be induced to maintain the internal balance. However, it is believed that the autophagy process may finally cause programmed cell death, if the stresses are continuously applied. The programmed cell death on the endothelial cells may influence severe and whole-body cell death. Hence, in the present invention, Con A can induce autophagy on the endothelial cells. Preferably, Con A induces LC3 and BNIP 3 conversion on endothelial cells.

In addition, the Con A-induced damage of hepatic endothelial cells causes the damage liver blood vessel, and the damage liver blood vessel occurs before the induction of T-cell-dependent hepatitis via apoptosis or T-cell-independent hepatitis via autophagy. According to the present invention, the autophagy on the endothelial cells can be observed at 3-4 h post-injection of Con A. In addition, the elevation of the serum alanine aminotransferase (ALT), which is caused by cytokine-caused apoptosis, is observed at 6 h post-injection, and then increased at 12-24 h.

With reference to the sensitivity to Con A, the direct autophagic induction needs a dose >30 mg/kg in murine model. Both endothelial cells and hepatocytes can be induced by Con A to undergo autophagy, but endothelial cells are more susceptible than hepatocytes. The hepatocyte damage can be either autophagy (high dose, 40 mg/kg in SCID/NOD mice) or apoptosis (low dose, 20 mg/kg in BALB/c mice).

When Con A binds to the endothelial cells, it causes autophagy or plasma leakage and hemorrhage. Hence, Con A can be used as a blood vessel destructing agent. The effect of the currently used blood vessel destructing agent, including small molecules or proteins synthesized by genetic engineering, is weaker than that of Con A. Hence, the present invention also provides a pharmaceutical composition for destructing blood vessels, which comprises an effective amount of Con A to induce damages of endothelial cells. Preferably, the blood vessels are hepatic blood vessels.

The blood vessel destructing agent is a new drug for treating a tumor, and it may cause tumor blood vessel collapse and tumor cell death. This has been reported in Baguley, 2003, Lancet Oncol 4: 141-148; Jassar et al., 2008, Drugs of the Future 33: 561-569; Cooney et al., 2006, Nat Clin Pract Oncol. 3: 682-692, and these papers are incorporated into the present invention. In addition, the blood vessel destructing agent does not have the problem of anti-drug, which the conventional anti-cancer drug usually has. The angiogenesis is a necessary process in the growth of the tumor cells. If the blood vessels in the tumor can be disrupted, it is possible to inhibit the growth of the tumor cells. In addition, it is believed that Con A deposited in the liver preferentially binds to hepatic sinusoidal endothelial cells, and induces the autophagy on the endothelial cells to cause the disruption of the blood vessels. Furthermore, the inflammatory cells recruit in the liver, and induce adaptive immune response on hepatic cells or liver tumor cells to cause hepatitis or tumor regression. In addition, Kupffer cells, NK cells, NKT cells, CD4⁺ T cells, and CD8⁺ T cells are activated, wherein CD8⁺ T cells are the most effective cells in killing tumor cells. The Con-A induced autophagy contributes to the antigen presentation of the liver tumor cells.

In addition, the pharmaceutical composition of the present invention can be in any conventional formulations including, but not limited to, injections, oral tablets, buccal tablets, oral solutions, and syrups. The pharmaceutical composition of the present invention comprises not only an effective amount of Con A, but also any essential adjuvants, excipients, or carriers. The components except the active components in the pharmaceutical composition of the present invention can be prepared by any conventional methods known in the art.

In addition, the pharmaceutical composition of the present invention can be administered through any conventional methods. For example, the pharmaceutical composition of the present invention can be formulated into an injection solution, and directly applied on the treatment position, including tumor cells or tumor tissue. Hence, Con A can directly induce damages of endothelial cells.

In addition, the pharmaceutical composition of the present invention can be orally administered. The pharmaceutical composition of the present invention can be transferred to the treatment position, through the digestive system or the circulatory system. For example, the oral pharmaceutical composition can be transferred from a stomach to liver through hepatic portal veins, and contact with liver tumor cells to achieve the effect of inducing damages of endothelial cells.

### BRIEF DESCRIPTION OF THE DRAWINGS

FIG. 1 is results showing that Con A-FITC binds to endothelial cells and hepatoma cells ML1, which is determined by flow cytometry;

FIGS. 2A to 2F are results showing that Con A induces hemorrhage before hepatitis in murine liver, wherein FIGS. 2A, 2C, and 2E show the liver injury evaluated by serum ALT level, and FIGS. 2B, 2D, and 2F show the hemorrhage status determined by Evans blue dye leakage from blood vessels;

FIGS. 3A to 3C are results showing Con A induces autophagy on HMEC-1 cell; and

FIGS. 4A to 4B are results showing IFN-γ enhances the Con A-mediated hemorrhage in liver.

### DETAILED DESCRIPTION OF THE PREFERRED EMBODIMENT

### Material and Method

### Mice

Eight- to 10-week-old male BALB/c, SCID/NOD (non-obese diabetic) and C57BL/6 mice were from National Cheng Kung University laboratory animal center. B6.129S7-Ifngtm1Ts/J (IFNG^{-/-}) and B6.129S7-Ifngr1tm1Agt/J (IFNGR^{-/-}) mice were from Jackson Laboratories (Bar Harbor, Me., USA). The mice were maintained in NCKU laboratory animal center. The animals were raised and cared for according to the guidelines set up by the National Science Council, Republic of China. The mouse experiments were approved by the Institutional Animal Care and Use Committee. To induce hepatitis, mice were injected intravenously with various doses of Con A, and the serum was collected at various time points post-injection. The activities of serum alanine aminotransferase (ALT) and serum aspartate aminotransferase (AST) were determined by Hitachi type 717 automatic analyzer (Hitachi) (Chang and Lei, 2008, Int. J. Immunopharmaco. Pharmacol. 21: 817-26).

### Con A Binding to Endothelial Cells

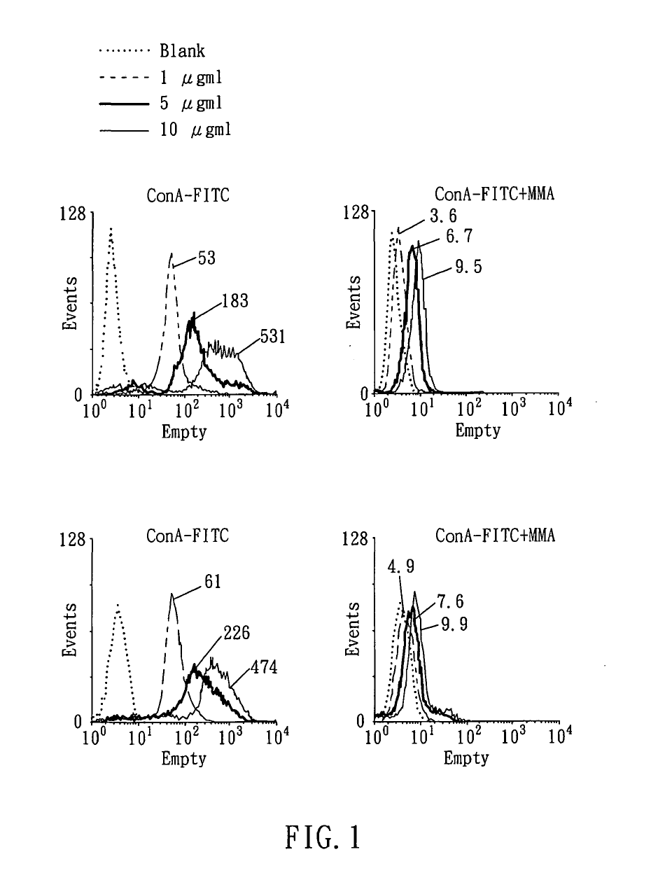

Human endothelial cell line HMEC-1 and mouse hepatoma cell line ML1 were incubated with different doses of Con A-FITC (Sigma, St Louis, Mo., USA) (1-10 μg/ml) at 37° C. for 30 min, after being washed by PBS, and the Con A binding activity was then determined by FACS Calibur. The binding can be blocked by adding 125 mM methyl-α-Dmannopyranoside (MMA) (Sigma) mixed with Con A-FITC to the cells. The results are shown in FIG. 1.

For the in vivo experiment, Con A-FITC (10 mg/kg) was intravenously injected to BALB/c mice, and at 1-6 h post-injection, the mice livers were harvested to investigate the Con A binding feature. The harvested livers were perfused by 10 ml of 4% paraformaldehyde to fix the binding. The frozen liver tissue section was then further fixed by 3.7% formaldehyde and subsequently stained with anti-CD31 antibody (BD Bioscience, San Jose, Calif., USA) and anti-rat conjugate Alexa 594 (Invitrogen, Carlsbad, Calif., USA) to demonstrate endothelial cells.

Blood Vessel Leakage Determination with Evans Blue and BSA-Fluorescein (FITC-BSA)

BALB/c, SCID/NOD, C57BL/6, B6.129S7-Ifngtm1Ts/J (IFNG^{-/-}) and B6.129S7-Ifngr1tm1Agt/J (IFNGR^{-/-}) mice treated with or without Con A were intravenously injected 50 mg/kg Evans blue dye in Dulbecco's Phosphate Buffered Saline (DPBS) (Invitrogen) at 30 min prior to killing. The liver tissue was perfused with 10 ml PBS, then formamide (4 ml/g tissue) was added and further incubated at 37° C. for 48 h. The liver tissue was centrifuged at 3000 r.p.m. for 10 min, and the supernatant was harvested to determine the blood vessel leakage by detection OD 630 (nm) of spectrophotometer.

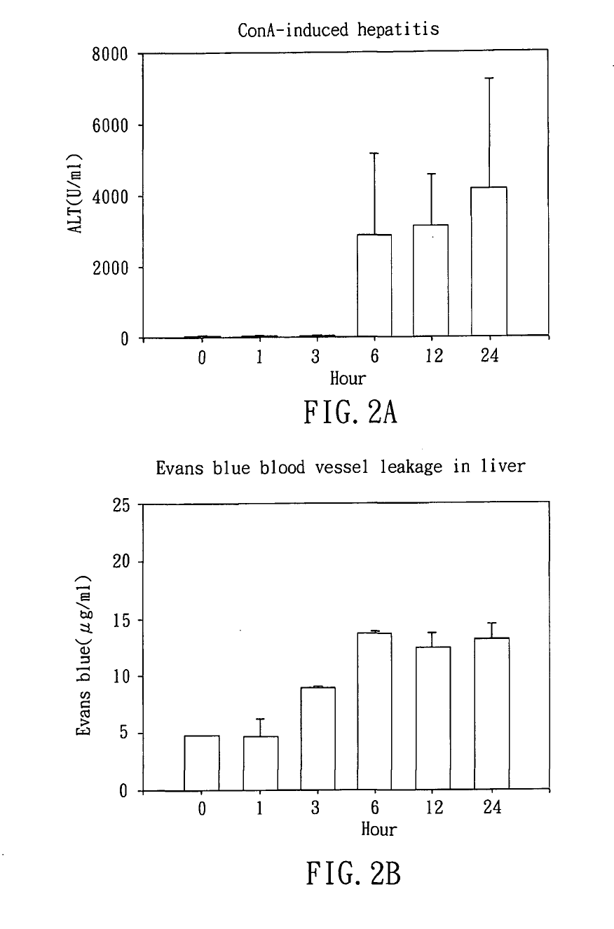

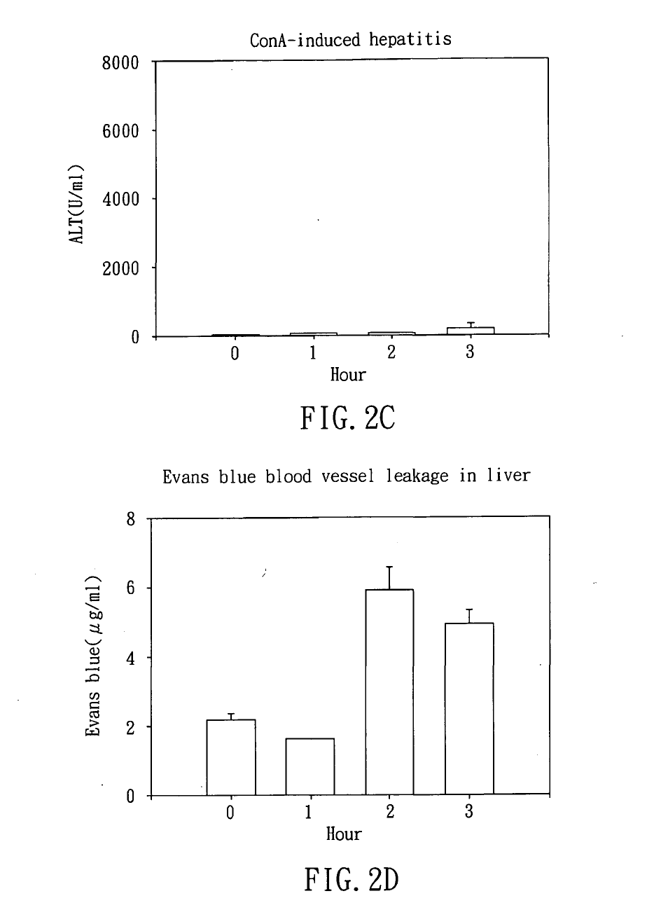

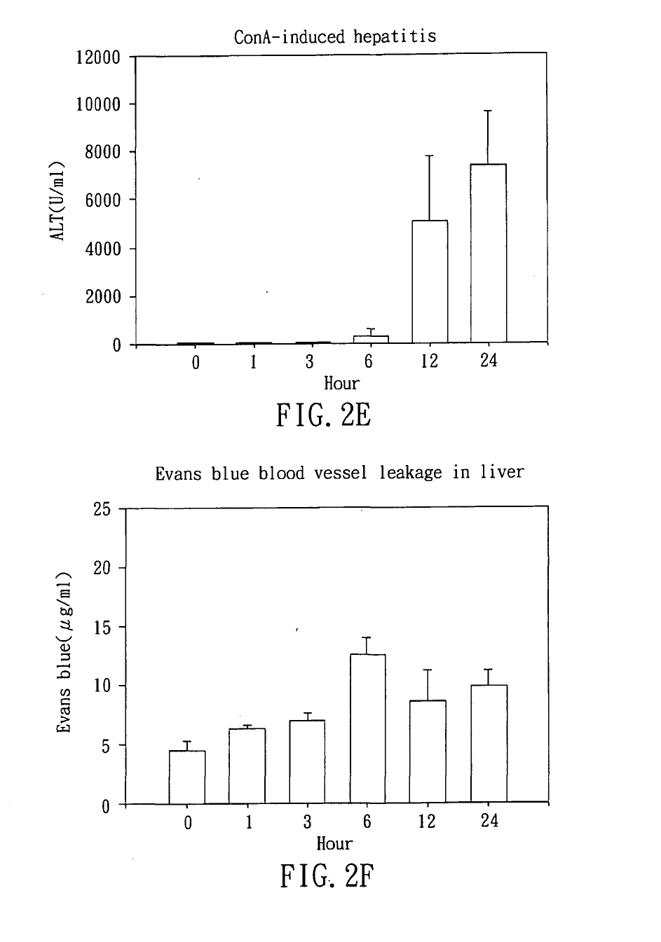

Mice treated with or without Con A were intravenously injected 10 mg/kg FITC-BSA (green color) in DPBS, liver tissue was perfused with 10 ml PBS, then stained with anti-CD31 antibody (red color). The blood vessel leakage was determined by a fluorescence microscopy. The results are shown in FIGS. 2A to 2F.

### Immunohistochemical Staining for Autophagy and Apoptosis in Endothelial Cells

The frozen liver tissue sections were stained with anti-LC3 antibody (Abgent, San Diego, Calif., USA), anti-rabbit conjugate Alexa 594 (Invitrogen), anti-CD31 and anti-rat conjugate Alexa 594 (Invitrogen) to determine the autophagy induction in endothelial cells. For the staining of Transferase dUTP nick end labeling (TUNEL-positive) apoptotic cells, the formalin-fixed and paraffin-embedded liver tissue sections were stained with the ApoAlert DNA Fragmentation Assay Kit (Clontech Laboratories, Mountain View, Calif., USA). The experimental procedure was proceeded according to the manufacturer's instructions.

### Cell Viability Determination

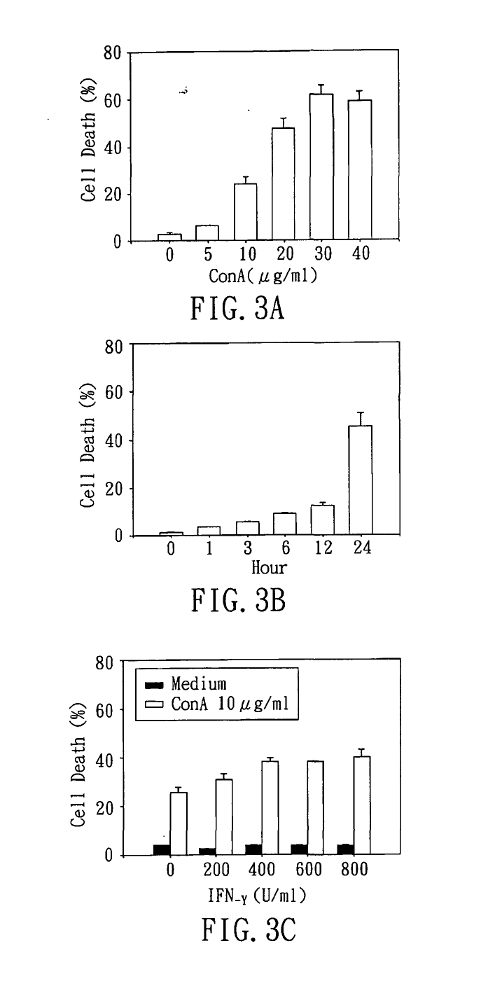

The HMEC-1 cell viability was determined by propidium iodide (PI) staining. The cells were washed by PBS once and re-suspended in 5 μg/ml PI, incubated at room temperature for 10 min, and then analyzed by FACScan. The PI-positive cells were considered to be dead. The cell death was determined with flow cytometry, and the results are shown in FIGS. 3A to 3C.

### Western Blotting

HMEC-1 cells were treated with different doses of Con A with or without IFN-γ (PeproTech, Rocky Hill, N.J., USA), and total cell protein was obtained by incubation in lysis buffer (Cell Signaling Technology, Danvers, Mass., USA). Proteins were separated by 12% SDS PAGE and transferred to PVDF membranes. The membranes were blocked with 5% skim milk and incubated with primary antibodies, including LC-3 (Abgent), BNIP3 (BD), BECLN (Santa Cruz Biotechnology) and β-actin (Chemicon), at 4° C. overnight. After incubation with peroxidase conjugated secondary antibodies at room temperature for 2 h, the blots were visualized by enhancing chemiluminescence reagents (PerkinElimer, Waltham, Mass., USA).

### Result

### Con a Preferentially Binds to Hepatic Sinusoidal Endothelial Cells Before the Induction of Hepatic Inflammation

FIG. 1 shows that human endothelial cell line HMEC-1 and mouse hepatoma cell line ML1 cells were treated with 1, 5 and 10 μg/ml Con A-FITC at 37° C. for 30 min in vitro. The upper two diagrams are results of HMEC-1, and the lower two diagrams are results of ML1. This in vitro binding activity was determined by flow cytometry.

According to the results shown in FIG. 1, when the human endothelial cell line HMEC-1 was used, it was found that Con A can specifically bind to endothelial cells dose dependently. The ML1 hepatoma cell line was used as a positive control. In addition, the binding can be blocked by MMA, indicating the mannose specificity of Con A.

According to the results of the in vivo binding experiment, after intravenous injection of Con A-FITC into BALB/c mice, Con A-FITC was found to deposit on the hepatic sinusoidal endothelial cells as identified by anti-CD31 antibody staining at 1 h post-injection. The endothelial cell surface molecule CD31 was co-localized with the Con A-FITC, and this result indicated that Con A preferentially binds to endothelial cells. With passage of time, the Con A-positive staining on endothelial cells gradually decreased, probably due to the internalization of Con A and degradation after binding to the cell membrane. Hence, most of the Con A-FITC were first preferentially bound to endothelial cells (1 h post-injection), but after some time (3 or 6 h post-injection), Con A bound to hepatocytes was observed. This result indicated that Con A was also internalized into hepatocytes.

### Con A-Induced Endothelial Cell Damage Before Hepatitis

FIGS. 2A, 2C, and 2E show the liver injury evaluated by serum ALT level, and FIGS. 2B, 2D, and 2F show the hemorrhage status determined by Evans blue dye leakage from blood vessels.

Since the lethal dose for immunocompetent BALB/c mice is at the dose >20 mg/kg (Chang and Lei, 2008, Int. J. Immunopharmaco. Pharmacol. 21: 817-26), both a non-lethal dose (20 mg/kg) and a lethal dose (30 mg/kg) of Con A are used to induce acute hepatitis. As shown in FIG. 2A, acute hepatitis was observed as early as 6 h, increasing gradually and peaking at 24 h post-intravenous injection. The endothelial cell damage in the liver was determined by using Evans blue as a plasma leakage marker from liver blood vessels. A significant leakage of Evans blue into the liver was observed at 3 h after Con A injection in BALB/c mice, as shown in FIG. 2B. In addition, the hematoxylin and eosin (H&E) stain of liver tissue also showed hemorrhage and necrosis in the liver. The red blood cells (RBCs) leaked into the tissue beginning at 3 h, becoming severe at 12-24 h that was associated with intense inflammation and necrosis, which is compatible with the data of Evans blue leakage.

A lethal dose of 30 mg/kg of Con A was also used to evaluate the effect of Con A on the hepatic blood vessels. The mice died within 5-6 h, and no significant hepatitis was observed at 3 h post-injection, as shown in FIG. 2C. However, hepatic blood vessel leakage and hemorrhage were observed at as early as 2 h post-injection, as shown in FIG. 2D. Although no increase of Evans blue was detected at 1 h, mild hemorrhage with RBC leakage was found at 1 h on H&E staining. These results indicate that Con A at 30 mg/kg causes more damage to hepatic blood vessels than at 20 mg/kg.

It is generally known that Con A induces acute hepatitis through the CD4⁺ T-cell-mediated inflammation that occurs at 6-24 h post-injection. Hemorrhage with blood vessel damage is associated with the inflammation and necrosis at this time point. However, in the present example, it is found that hepatic blood vessel damage and hemorrhage is induced at 2-3 h after Con A injection without lymphocyte infiltration and hepatitis.

It has been reported a T-cell-independent acute hepatitis in SCID/NOD mice (Chang and Lei, 2008, Int. J. Immunopharmaco. Pharmacol. 21: 817-26). Con A at 40 mg/kg was injected intravenously into SCID/NOD mice, and the hepatitis and hemorrhage were analyzed kinetically. No alanine transaminase elevation was observed until 12 h post-injection, as shown in FIG. 2E. However, Evans blue leakage was detected as early as 3 h, with a higher level at 6 h post-injection, and maintained a significant level at 12-24 h, as shown in FIG. 2H. In addition, the H&E stain of liver tissue confirmed the hemorrhage beginning at 3 and 6 h without the inflammatory cell infiltration. Necrosis was observed at a later time of 12-24 h. These results indicated that Con A can cause hemorrhage before the induction of hepatitis. The hepatic blood vessel can therefore be directly damaged by Con A in either immunocompetent or immunodeficiency mice.

### Con A-Induced Autophagy of Endothelial Cells

To further understand how the blood vessel is damaged by Con A to cause hemorrhage, liver tissue was stained with both autophagy and apoptosis markers. LC3-II punctate formation was observed on hepatic blood vessels of SCID/NOD mice at dose of 30 mg/kg, but not 20 mg/kg. A dose of 40 mg/kg showed more LC3-II punctates. When BALB/c mice were tested with the dose of 30 mg/kg of Con A, the autophagy LC3 markers were co-localized with the CD31⁺ endothelial cells at 4 h post-injection. On the other hand, no TUNEL-positive cells were detected in the liver at 3 h post-injection, whereas TUNEL-positive apoptosis was observed in hepatocytes at 12-24 h when 20 mg/kg of Con A was injected in BALB/c mice. Apparently, hepatic endothelial cells were stimulated by Con A to undergo autophagy but not apoptosis.

The signal pathway was further studied on the human endothelial cell HMEC-1. HMEC-1 cells were treated with 5, 10, 20, 30 and 40 μg/ml Con A for 24 h and stained with PI to determine the cell death using flow cytometry. The results are shown in FIG. 3A. In addition, time-dependent response was assayed with 20 μg/ml Con A at 1, 3, 6, 12 and 24 h post-treatment, and the results are shown in FIG. 3B. As shown in FIGS. 3A and 3B, these results indicate that Con A can induce dose- and time-dependent death of HMEC-1 cells. In addition, the autophagic marker of LC3 II conversion was also demonstrated by Western blot analysis. Hence, Con A induces autophagy on hepatic endothelial cells.

Con A Binding to Intra-Tumor Endothelial Cells and Inducing Plasma Leakage within Tumor

Con A-FITC (green) was intravenously injected into tumor bearing mice, and the harvested tissue sections were stained with anti-CD31 antibody to indicate the location of endothelial cells (red). It was found that a lot of Con A had bound to endothelial cells in normal liver tissue. On the other hand, the endothelial cell surface molecule CD31 within tumor co-localized with the Con A-FITC was less than that within normal tissue. Then, FITC-BSA was injected into tumor bearing mice, and the Con A-induced plasma leakage was observed. A little amount of FITC-BSA was found in normal liver tissue, and it indicated that a basic leakage was caused. The basic leakage in the normal liver tissue may be related to the structure of the sinusoid endothelial cells. However, at 3 h post-injection of Con A (20 mg/kg), the leakage from liver blood vessels in normal tissue was not increased, but the leakage from liver blood vessels in tumor tissue was increased greatly. In tumor tissue, FITC-BSA leakage can be observed within every CD31⁺ endothelial cells in the tumor bearing mice with Con A treatment, but it cannot be observed in the tumor bearing mice without Con A treatment. These results indicated that Con A can bind to the intra-tumor endothelial cells, induce damages of endothelial cells, and cause plasma leakage.

### IFN-γ Enhanced the Con A-Mediated Autophagy of Endothelial Cells

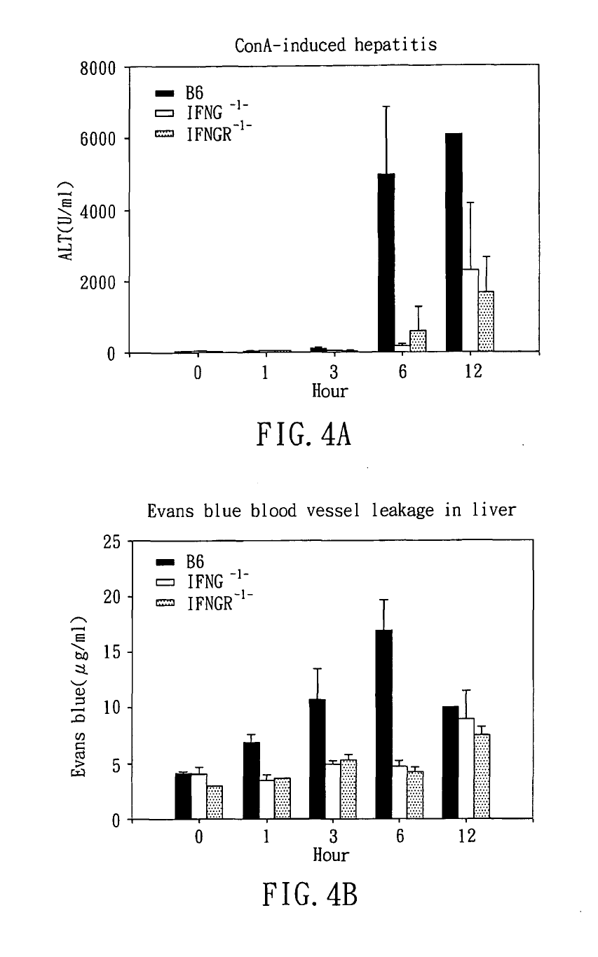

Con A at 30 mg/kg was injected into the IFN-γ and IFNR knockout mice, and the Con A-induced acute hepatitis was ameliorated in the knockout mice (IFNG^{-/-} or IFNGR^{-/-}) comparing with the wild type (B6). The results are shown in FIG. 4A. Moreover, there was low level of Evans blue extravasation on IFNGR^{-/-} mice. When comparing with the wild-type and IFN-γ and IFNR knockout mice, the IFN-γ significantly enhanced the Evans blue extravasation and hemorrhage with RBC leakage in hepatic blood vessels at 1-6 h after Con A injection, as shown in FIG. 4B.

On HMEC-1 cell, Con A-induced cell death can be enhanced by IFN-γ dose dependently, as shown in FIG. 3C. In addition, it was also found that autophagic LC3-II conversion from LC3-I was enhanced by IFN-γ. IFN-γ treatment seems to sustain the autophagy process by maintaining the level of LC3-I.

Based on the results above, it is proven that Con A induces autophagy on hepatic endothelial cells, which can be enhanced by IFN-γ, and this liver blood vessels damage occurs before the induction of hepatitis.

Although the present invention has been explained in relation to its preferred embodiment, it is to be understood that many other possible modifications and variations can be made without departing from the scope of the invention as hereinafter claimed.

What is claimed is:

1. A pharmaceutical composition for inducing damages of endothelial cells, comprising:
an effective amount of Concanavalin A (Con A).

2. The pharmaceutical composition as claimed in claim 1, further comprising at least one selected from the group consisting of adjuvants, excipients, and carriers.

3. The pharmaceutical composition as claimed in claim 1, wherein the endothelial cells are hepatic endothelial cells, or liver tumor endothelial cells.

4. The pharmaceutical composition as claimed in claim 1, wherein the endothelial cells are hepatic vascular endothelial cells, or liver tumor vascular endothelial cells.

5. The pharmaceutical composition as claimed in claim 1, wherein Con A induces autophagy on the endothelial cells.

6. The pharmaceutical composition as claimed in claim 1, wherein the effective amount of Con A is 20-40 mg/kg.

7. A pharmaceutical composition for treating a tumor, comprising:
an effective amount of Con A.

8. The pharmaceutical composition as claimed in claim 7, further comprising at least one selected from the group consisting of adjuvants, excipients, and carriers.

9. The pharmaceutical composition as claimed in claim 7, wherein Con A induces damages of endothelial cells.

10. The pharmaceutical composition as claimed in claim 7, wherein the tumor is a liver tumor.

11. The pharmaceutical composition as claimed in claim 8, wherein the endothelial cells are hepatic endothelial cells, or liver tumor endothelial cells.

12. The pharmaceutical composition as claimed in claim 8, wherein the endothelial cells are hepatic vascular endothelial cells, or liver tumor vascular endothelial cells.

13. The pharmaceutical composition as claimed in claim 8, wherein Con A induces autophagy on the endothelial cells.

14. The pharmaceutical composition as claimed in claim 7, wherein the effective amount of Con A is 20-40 mg/kg.

15. A method for treating a tumor, comprising:
administering a pharmaceutical composition to a patient, wherein the pharmaceutical composition comprises an effective amount of Con A.

16. The method as claimed in claim 15, wherein the pharmaceutical composition further comprises at least one selected from the group consisting of adjuvants, excipients, and carriers.

17. The method as claimed in claim 15, wherein Con A induces damages of endothelial cells.

18. The method as claimed in claim 15, wherein the tumor is a liver tumor.

19. The method as claimed in claim 17, wherein the endothelial cells are hepatic endothelial cells, or liver tumor endothelial cells.

20. The method as claimed in claim 17, wherein the endothelial cells are hepatic vascular endothelial cells, or liver tumor vascular endothelial cells.

21. The method as claimed in claim 17, wherein Con A induces autophagy on the endothelial cells.

22. The method as claimed in claim 18, wherein the effective amount of Con A is 20-40 mg/kg.
